(19) United States
(12) Patent Application Publication
(10) Pub. No.: US 2004/0000314 A1
(43) Pub. Date: Jan. 1, 2004

(54) Airway assembly

(76) Inventor: Luis F. Angel, San Antonio, TX (US)

Correspondence Address: ERIC B. MEYERTONS, MEYERTONS, HOOD, KIVLIN, KOWERT & GOETZEL, P.C., P.O. BOX 398, AUSTIN, TX, 78767-0398, US

(21) Appl. No.: 10/396,135

(22) Filed: Mar. 20, 2003

Related U.S. Application Data
(60) Provisional application No. 60/365,885, filed on Mar. 20, 2002.

(51) Int. Cl. A61M 16/00; A61M 15/08

(52) U.S. Cl. 128/207.14; 128/207.16; 128/207.18

(57) ABSTRACT

An airway assembly may be used in a procedure that requires instrumentation to be inserted in an air passage of a patient. The airway assembly may include a reinforced and flexible first conduit, an expandable member, a second conduit and a third conduit. The second and third conduit may couple to an outer surface of the first conduit. Coupling the second and third conduits to an outer surface of the first conduit may allow the first conduit to have an unobstructed lumen that facilitates insertion of instruments into a patient. Fluid may be removed through the second conduit. Ventilation of a patient may be performed through the third conduit. The expandable member may seal against the air passage and inhibit fluid from passing from a first region to a second region and/or inhibit fluid from passing from the second region to the first region.

### PRIORITY CLAIM

This application claims priority to Provisional Patent Application No. 60/365,885 entitled “AIRWAY ASSEMBLY” filed on Mar. 20, 2002.

### BACKGROUND

1. Field of the Invention

The present invention generally relates to an airway assembly. Embodiments of the invention relate to an apparatus that provides for ventilation of a patient, suctioning of fluids, and manipulation of instrumentation inserted in an air passage of the patient.

2. Description of Related Art

During some surgical procedures, apparatus and/or instrumentation may be inserted through the mouth or nose and positioned in an air passage of a patient. As used herein, “air passage” refers to a patient's larynx, trachea, bronchi or bronchioles. The apparatus and/or instrumentation may be used to ventilate, remove fluid (e.g., bodily fluids and/or gases), observe, treat and/or obtain biological material from within the patient. In some patients, insertion of the apparat us and/or instrumentation is difficult due to structural variances in the patient's air passage (e.g., a patient may have a large tongue, swelling and/or excessive pharyngeal or laryngeal soft tissue). Physiological events such as a laryngospasm, regurgitation of gastric materials, bleeding or foreign body aspiration in the air passage may make surgical procedures difficult.

An example of an apparatus inserted in an air passage is a tube used for ventilation of a patient. An oxygen-containing gas is supplied through the tube during ventilation. By-products of respiration may be allowed to leave the patient through the air passage. Techniques for ventilation include, but are not limited to, manual ventilation, jet ventilation and high frequency ventilation. Ventilating systems may include, but are not limited to, additional components that aid in monitoring vital signs of the patient during the surgical procedure. Examples of patient vital signs include blood pressure, pulse and temperature.

U.S. Pat. No. 4,584,998 to McGrail, which is incorporated by reference as if fully set forth herein, describes a multi-purpose tracheal tube for use with high frequency ventilation. The tracheal tube includes a primary tube with three smaller lumens located in the tube wall of the primary tube.

An endoscopic system is an example of an apparatus and an instrument that may be inserted in an air passage. An endoscopic system may include a scope and multiple tubes. The scope may be used to visualize a desired affected area of the air passage and/or an area beyond or adjacent to the air passage. The tubes may be used to ventilate the patient and/or provide a channel for manipulation of surgical instrumentation.

U.S. Pat. No. 6,086,529 to Arndt, which is incorporated by reference as if fully set forth herein, describes a bronchoscopic manifold. The manifold may consist of a hollow elongated manifold for fiberoptic bronchoscopy. A secondary tubular instrument may be inserted through a threaded secondary instrument port. Ventilation of the intubated patient is performed by use of a mechanical ventilator connected to a ventilation port and an endotracheal tube connection port.

An apparatus inserted in an air passage may provide for removal of bodily fluids, such as blood, mucus and gastric fluids. U.S. Pat. No. 5,143,062 to Peckham, which is incorporated by reference as if fully set forth herein, describes an endotracheal tube that may suction a patient's secretions. The endotracheal tube may include a double lumen and an inflatable cuff. The double lumens may be formed in the wall of the tube and are used for evacuating an area above the inflatable cuff.

An instrument inserted in a patient may include an expandable member. The expandable member may be expanded against a portion of the body or another instrument to form, or substantially form, a seal. The seal may inhibit passage of fluid past the expandable member. U.S. Pat. No. 5,520,175 to Fry and U.S. Pat. No. 5,295,489 to Bell et al., both of which are incorporated by reference as if fully set forth herein, describe expandable members.

### SUMMARY

Instruments may be inserted into a patient through an airway assembly inserted in an air passage of the patient. An airway assembly may include a reinforced and flexible first conduit, a second conduit (e.g., suction conduit), a third conduit (e.g., ventilation conduit) and an expandable member. The second and third conduits may couple to an outer surface of the first conduit. Coupling the second and third conduits to an outer surface of the first conduit may allow the first conduit to have a large and unobstructed lumen that facilitates insertion of instruments into a patient. As used herein, “lumen” refers to a cavity or channel within a conduit. The expandable member may be expanded against a wall of an air passage to separate a first region of the patient from a second region of the patient. The expandable member may inhibit fluid from passing from the first region to the second region and/or inhibit fluid from passing from the second region to the first region.

A first conduit of an airway assembly embodiment may be reinforced to provide structural strength and inhibit collapse of the first conduit. Reinforcement for the first conduit may be, but is not limited to, a spiral of structural material in a body of the first conduit, a ring or rings of structural material located in a body of the first conduit at a position or positions along a length of the first conduit. The structural material may be, but is not limited to, metal, a metallic alloy, polymers or combinations thereof. Portions of the first conduit may be flexible to allow the airway assembly to conform to curves of an air passage during insertion of the airway assembly in an air passage of the patient.

An airway assembly embodiment may include a second conduit. A portion of the second conduit may be coupled to an outer surface of a first conduit of the airway assembly. A portion of the second conduit may be flexible to facilitate insertion of the airway assembly into a patient. A portion of the second conduit may pass through an expandable member of the airway assembly. Pressure of the region below the expandable member may be reduced through the second conduit to provide suction to the region below the expandable member for removal of fluids from the region. The second conduit may include reinforcement that inhibits collapse of the second conduit. In an embodiment of an airway assembly, a portion of a second conduit that passes through an expandable member of the airway assembly may be a metallic and/or a reinforced conduit. The metallic and/or reinforced conduit may inhibit collapse of an air passage of a patient when the expandable member is activated to seal against a wall of the air passage.

An airway assembly embodiment may include a third conduit. A portion of the third conduit may be coupled to an outer surface of a first conduit of the airway assembly. A portion of the third conduit may be flexible to facilitate insertion of the airway assembly in an air passage of a patient. An opening in the third conduit, or an end of the third conduit, may be located above an expandable member of the airway assembly. After expansion of the expandable member to seal against an air passage, the third conduit may be used to ventilate a patient.

Monitoring equipment may be coupled to conduits of an airway assembly to monitor the status of a patient. For example, pressure monitors, carbon dioxide monitors and/or temperature probes may be coupled to conduits of an airway assembly. During use of some airway assemblies, medication may be directed to a desired region. For example, medication may be directed to an upper region of a patient through a third conduit of an airway assembly. Medication may be directed to a lower region of a patient through a second conduit and/or a first conduit of the airway conduit.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a side representation of an embodiment of an airway assembly;

FIG. 2 depicts a cross-sectional representation of an embodiment of an airway assembly, taken substantially along line 2-2 of FIG. 1;

FIG. 3 depicts a cross-sectional representation of a portion of an embodiment of an airway assembly embodiment;

FIG. 4 depicts a representation of a portion of an airway assembly embodiment with an uninflated expandable member in an air passage; and

FIG. 5 depicts a representation of a portion of an airway assembly embodiment with an inflated expandable member in an air passage.

While the invention may be susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION OF EMBODIMENTS

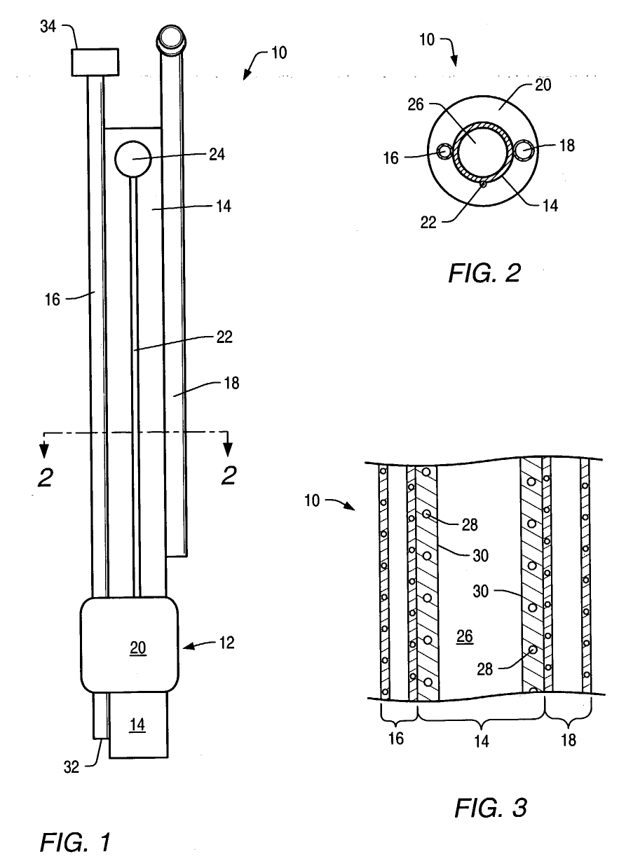

Referring to the drawings, and particularly to FIG. 1 and FIG. 2, an airway assembly is designated generally by reference number 10. Airway assembly 10 may be used in a surgical procedure that requires an apparatus and/or instrumentation to be inserted in an air passage of a patient. Examples of surgical procedures may include ventilating, suctioning fluid, observing, treating and/or obtaining biological material from within the patient. Instruments may be, but are not limited to, endoscopes, biopsy needles, forceps, cutters and/or large volume suction tubes. The term “biopsy” refers to a procedure that involves obtaining a tissue sample and/or body fluids from a patient.

Airway assembly 10 may be made of various materials include, but are not limited to, metals, metal alloys, silicon, plastic, polymers, ceramics and combinations thereof. Some airway assembly embodiments may include components made of materials that can be autoclaved and/or chemically sterilized. Some components of an airway assembly may be formed of materials unable to be autoclaved and/or chemically sterilized. Components unable to be autoclaved and/or chemically sterilized may be made of sterile materials and placed in working relation to other sterile components during assembly of an airway assembly. In some embodiments, an entire airway assembly may be made of materials that can be autoclaved and/or chemically sterilized so that the airway assembly is a reusable instrument. In other airway assembly embodiments, all or selected components of the airway assembly may be made of sterile, disposable materials so that all or components of the airway assembly are designed for single use.

An embodiment of airway assembly 10 may include expandable member 12, first conduit 14, second conduit 16 and third conduit 18. Airway assembly 10 may be inserted in an air passage of a patient. Expandable member 12 may be activated to expand against the air passage. Expandable member 12 may seal against a wall of the air passage. The seal between the air passage wall and expandable member 12 may inhibit passage of fluid past the expandable member. Expandable member 12 may define a first region on a proximal side of the expandable member and a second region on a distal side of the expandable member.

Expandable member 12 may include inflation member 20, inflation conduit 22, and inflator 24. Inflation member 20 and inflation conduit 22 may be coupled to first conduit 14. In an expandable member embodiment, inflation member 20 is a balloon. When inflation member 20 is in a deflated state, airway assembly 10 may be inserted in an air passage of a patient. Portions of inflation member 20 and inflation conduit 22 may be flexible to allow for insertion of airway assembly in an air passage of the patient. After insertion, inflation member 20 may be inflated to press against a wall of the air passage. Inflation member 20 may form a seal against the wall of the air passage. Formation of the seal may inhibit fluid flow past the inflation member and form two regions in the patient.

Inflation member 20 may be inflated or deflated using inflation conduit 22 and inflator 24. Inflation conduit 22 may transport an inflation fluid (e.g., a gas or a liquid) into inflation member 20. The fluid may be supplied from inflator 24. Inflator 24 may be, but is not limited to, a bellows, a pump and/or a fluid supply. After inflation, fluid flow may be inhibited out of inflation member 20 through generally known techniques. In an embodiment, a valve may be turned to inhibit flow of fluid out of inflation member 20. To deflate inflation member 20, the valve may be turned to allow fluid to flow out of the inflation member. In certain expandable member embodiments, constant pressure may be applied to inflation member 20 to inhibit flow of fluid out of the inflation member. To deflate inflation member 20, the pressure applied to inflation member may be terminated to allow fluid to flow out of the inflation member. In some expandable member embodiments, an inflator may operate in a first way to allow fluid to be inserted into an inflation member. The inflator may operate in a second way to allow fluid to be removed from the inflation member. In some expandable member embodiments, an inflator may be attached to inflation conduit to fill expandable member. The inflator may be removed from the inflation conduit to allow the inflation member to be deflated. In some embodiments, a deflator may be attached to the inflation conduit to remove fluid from the inflation member to deflate the inflation member.

In some airway assembly embodiments, instruments may be inserted through lumen 26 (depicted in FIG. 2) of first conduit 14. Instruments may be used to observe or affect tissue or other matter in a second region distal to expandable member 12. In some embodiments, second conduit 16 may be used as a suction line to remove fluid from the second region. In some embodiments, third conduit 18 may be used as a ventilation line for supplying respiration gases to a first region proximal to expandable member 12.

FIG. 2 depicts a cross-sectional representation of an embodiment of an airway assembly taken substantially along line 2-2 of FIG. 1. As depicted in FIG. 2, second conduit 16 and third conduit 18 may be coupled to an outer surface of first conduit 14 so that lumen 26 is large and unobstructed. A large and unobstructed first conduit lumen may facilitate insertion of instruments into the first conduit. Adhesive, straps, wrapping and/or tape may be used to couple portions of second conduit 16 and third conduit 18 to first conduit 14. For example, the second conduit and the third conduit may be taped to the first conduit. In some airway assembly embodiments, a portion of second conduit 16 and a portion of third conduit 18 may be welded, sonically welded or molded to first conduit 14. Other methods may also be used to couple a portion of a second conduit and/or a portion of a third conduit to the first conduit. It should be understood that first conduit 14, second conduit 16 and third conduit 18 do not become separated or spaced-apart from each other along the portion that is “coupled” during a surgical procedure.

First conduit 14 may be made of a material or materials that allow a portion of the first conduit to be flexible. A flexible first conduit may facilitate insertion of airway assembly 10 into a patient's air passage. A portion of first conduit 14 may include reinforcement 28 that inhibits collapse of the first conduit. As depicted in FIG. 3, reinforcement 28 may be present in wall 30 of first conduit 14. Reinforcement 28 may be, but is not limited to, a spiral of reinforcing material, rings of reinforcing material or combinations thereof. The reinforcing material may include, but is not limited to, metallic wire, polymer strands and/or polymer and wire combinations. Reinforcement 28 may inhibit collapse of first conduit 14 while allowing for some flexibility of the first conduit. Reinforcement 28 may inhibit collapse of the patient's air passage during a surgical procedure.

A portion of first conduit 14 may include a layer or coating of a material on an inner surface that facilitates insertion of instruments into the first conduit. For example, the inner surface of first conduit 14 may include a fluorine-containing resin layer (e.g., TEFLON®) or other material with a low coefficient of friction. A portion of first conduit 14 may include a layer or coating of a material on an outer surface that facilitates insertion of airway assembly 10 in an air passage.

A portion of first conduit 14 may be sized to fit within an air passage of a patient. The first conduit may be cylindrical in shape. First conduit 14 may have a diameter between about 3 mm and about 20 mm. In some embodiments, first conduit 14 may have a diameter between about 10 mm and about 17 mm. First conduits having larger or smaller diameters may be used to accommodate specific requirements of a patient.

Second conduit 16 may be used to remove fluids from a region distal to an expanded inflation member 20. Removal of fluids through second conduit 16 may be performed by reduction of pressure (e.g., suction) distal to inflation member 20 using known methods (e.g., vacuum or partial vacuum). In some embodiments, second conduit 16 may be sized to accommodate a suctioning volume rate of about 500 cc/sec (cubic centimeters per second). In some embodiments, second conduit 16 may be cylindrical in shape. Cylindrical second conduit 16 may have a diameter between about 0.1 mm and about 3.0 mm. In some embodiments, second conduit 16 may have a diameter between about 0.5 mm and about 2.5 mm. Second conduits having larger or smaller diameters may be utilized to accommodate specific requirements of a patient.

Second conduit 16 may be reinforced to inhibit collapse of the conduit when pressure is reduced through the second conduit (e.g., when a vacuum is drawn). A portion of a second conduit 16 may pass through inflation member 20. The portion of second conduit 16 that passes through inflation member 20 may be a metallic section or a reinforced section to inhibit collapse of the portion when inflation member 20 is expanded against a wall of an air passage.

As shown in FIG. 1, distal end 32 of second conduit 16 may extend beyond inflation member 20. In some airway assembly embodiments, distal end 32 may extend to an end of first conduit 14. In some airway assembly embodiments, distal end 32 may terminate between about 0.1 cm and about 3.0 cm of an end of first conduit 14. In an embodiment, distal end 32 may terminate about 0.5 cm from the end of first conduit 14.

Portions of second conduit 16 may be flexible to facilitate insertion of airway assembly 10 into an air passage of a patient. An outer surface of second conduit 16 may include a layer or coating of a material having a low coefficient of friction to facilitate insertion of the second conduit into the air passage.

Proximal end 34 of second conduit 16 may be coupled to a pressure reduction source that reduces the pressure through the second conduit to remove fluids distal to expanded inflation member 20. Removal of fluids may allow for visualization of a procedural area. Removal of fluids may facilitate placement and/or manipulation of instruments in the procedural area.

During some surgical procedures, proximal end 34 of second conduit 16 may be coupled to a supply line. The supply line may allow rinse fluid, medication or other fluid to be inserted into a region beyond inflation member 20. If needed, suction distal to inflation member 20 may be provided through a suction conduit inserted into first conduit 14 when second conduit 16 is not coupled to a pressure reduction source (e.g., vacuum pump or aspirator).

Third conduit 18 may be used to ventilate a patient proximal to expanded inflation member 20. In some airway assembly embodiments, ventilation at a pressure between about 30 pounds per square inch (psi) and about 50 psi, with a volume between about 800 cc and about 1000 cc of oxygen, may be provided through third conduit 18. In some embodiments, third conduit 18 may be made of materials able to withstand over 100 psi. In certain embodiments, third conduit 18 may be cylindrical in shape. Third conduit 18 may have a diameter between about 0.1 mm and about 3.0 mm. In some embodiments, third conduit 18 may have a diameter between about 0.5 mm and about 2.5 mm. A third conduit with a larger or smaller diameter may be utilized to accommodate specific requirements of a patient.

In some airway assembly embodiments, such as the embodiment depicted in FIG. 1, third conduit 18 may end proximal to inflation member 20. In other airway assembly embodiments, a third conduit may extend to, or into, an inflation member. A portion of the third conduit may include a slot, slots or other types of openings that allow fluid to pass into or out of the third conduit.

Portions of third conduit 18 may be flexible to facilitate insertion of airway assembly 10 in an air passage of a patient. An outer surface of the third conduit may include a layer or coating of a material having a low coefficient of friction to facilitate insertion of the third conduit into the air passage.

Fluid may be delivered to a region that is proximal to inflation member 20. In an embodiment of airway assembly 10, fluid may be delivered through third conduit 18 to a region proximal to inflation member 20. The fluid may include, but is not limited to, lidocaine or other airway anesthesia, antifungals, gene therapy, antibiotics, immunosuppressants and/or chemotherapy drugs.

Third conduit 18 and/or second conduit 16 may be coupled to a monitor or monitors for determining patient status. Monitors may include, but are not limited to, pressure gauges, capnography monitors for CO₂ analysis and/or respiration monitors. In some embodiments, one or more temperature sensors may be coupled to first conduit 14, second conduit 16, and or third conduit 18 of airway assembly 10.

In some airway assembly embodiments, second conduit 16 and third conduit 18 may be positioned substantially 180° apart around a circumference of first conduit 14. Locating the second conduit and the third conduit at opposite positions relative to the first conduit may facilitate manipulation of connections to the second conduit and the third conduit. A proximal end of the second conduit may have a different connector than a proximal end of the third conduit connector to avoid confusion as to the identity of the conduits. For example, second conduit 16 may have a male connector that attaches to other equipment. Third conduit 18 may have a female connector that attaches to other equipment. To further distinguish between the conduits, second conduit 16 and third conduit 18 may be different colors (e.g., blue, green, red, yellow, orange). In some embodiments, second conduit 16 and third conduit 18 may be located at positions that are not substantially 180° apart around a circumference of first conduit 14.

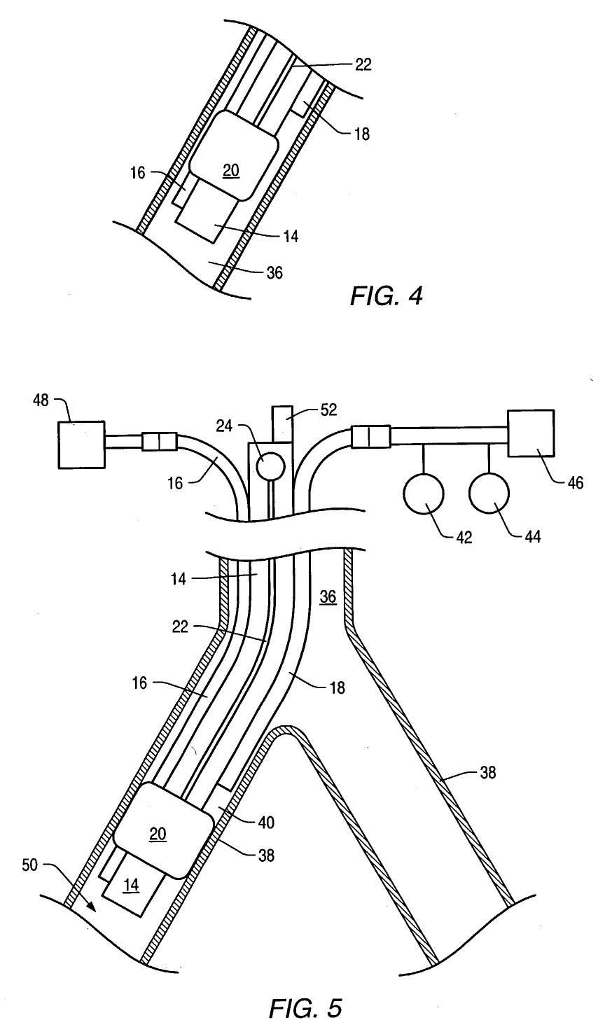

FIG. 4 depicts a representation of a portion of an embodiment of an airway assembly with an uninflated inflation member 20 positioned in air passage 36 of a patient. Air passage 36 may be a bronchus. After insertion of the airway assembly in air passage 36, inflation member 20 may be inflated through inflation conduit 22.

FIG. 5 depicts a representation of a portion of an embodiment of an airway assembly with an inflated inflation member 20 positioned in air passage 36 of a patient. Outer surface of inflation member 20 may press against wall 38 of air passage 36. Pressing the outer surface of inflation member 20 against wall 38 may form a seal between the inflation member and the wall. Formation of a seal may inhibit passage of fluid past the inflation member through air passage 36. Fluid may flow past inflation member 20 through second conduit 16 and/or through first conduit 14.

After inflation member 20 is inflated, first region 40 of air passage 36 proximal to the inflation member may be ventilated using third conduit 18. Pressure gauge 42, capnography monitor 44, and/or ventilator 46 may be coupled to third conduit 18. Pressure reduction source 48 (e.g., a vacuum pump) may be coupled to second conduit 16 to provide suction in second region 50 of air passage 36 distal to inflation member 20. Instrument 52 may be inserted into first conduit 14 to observe and/or manipulate tissue or fluid in second region 50. In some embodiments, airway anesthesia may be delivered to second region 50 through second conduit 16.

After manipulation in second region 50 is completed, an instrument or instruments may be removed from first conduit 14. Second conduit 16 and/or third conduit 18 may be decoupled from equipment such as pressure reduction source 48 and ventilator 46. Inflation member 20 may be deflated. Airway assembly 10 may be removed from air passage 36.

To form an airway assembly embodiment, an expandable member, a first conduit, a second conduit and a third conduit may be removed from an airway assembly package. The expandable member may include, but is not limited, an inflation conduit, an inflation member and an inflator. In certain embodiments, an inflation member may be a balloon cuff. The expandable member, the second conduit and the third conduit may be coupled to the first conduit to form an airway assembly. In some embodiments, an expandable member, a first conduit, a second conduit and a third conduit may be provided as a unit. In certain embodiments of an airway assembly, an expandable member, a second conduit and a third conduit may be removably coupled to a first conduit.

An airway assembly may be inserted in an air passage of a patient. After insertion in an air passage of a patient, a third conduit may be connected to a ventilation system, a capnography monitor and/or one or more temperature sensors. A second conduit may be connected to a pressure reduction source to provide suction to a region distal to the expandable member. An inflator may be coupled to a fluid source. Fluid may be delivered from the inflator, through an inflation conduit, to inflate an inflation member. After inflation, the inflation member may substantially block the air passage. After inflation of the inflation member, the inflator may be positioned to inhibit flow of the fluid out of the inflation member (e.g., a valve may be turned).

One or more instruments (e.g., a bronchoscope, a biopsy needle) may be inserted in a portion of the first conduit to visualize and/or operate on a region of the air passage proximal of the expandable member. In certain embodiments, an anesthetic may be delivered to the region proximal the expandable member through the third conduit. In some embodiments, an anesthetic may be delivered to a region in an air passage proximal to an expandable member through a third is conduit prior to insertion of one or more instruments in a first conduit. In other embodiments, a first instrument (e.g., bronchoscope) may be inserted in a portion of the first conduit to visualize a region proximal of an expandable member. A second instrument (e.g., biopsy needle) may, be inserted through a first instrument or a first conduit to remove bodily fluids and/or tissue from the region proximal the expandable member. In some embodiments, during a procedure, an anesthetic and/or medication may be delivered through a third conduit to the region proximal an expandable member.

After visualization and/or removal of tissue, one or more instruments may be removed from the first conduit. The inflator may be positioned to remove fluid through the inflation conduit to deflate the inflation member. The second conduit may be disconnected from a pressure reduction source and/or other instrumentation. The third conduit may be disconnected from the a ventilation source. After deflation of the inflation member and/or disconnection of the second conduit and the third conduit, the airway assembly may be removed from an air passage of a patient. The expandable member, the second conduit and the third conduit may be decoupled from the first conduit. Decoupling of the expandable member, the second conduit and the third conduit from the first conduit may allow for reuse of the individual components. In some embodiments, an airway assembly may be disposed of as a unit.

In this patent, certain U.S. patents have been incorporated by reference. The text of such U.S. patents is only incorporated by reference to the extent that no conflict exists between such text and the other statements and drawings set forth herein. In the event of such conflict, then any such conflicting text in such incorporated by reference U.S. patents is specifically not incorporated by reference in this patent.

Further modifications and alternative embodiments of various aspects of the invention will be apparent to those skilled in the art in view of this description. Airway assemblies may be modified to operate in other areas of a patient in which it is desired to separate a first region from a second region by a seal formed in a passage of the patient. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. An airway assembly comprising:
a first conduit comprising reinforcement to inhibit collapse of the first conduit;
an expandable member coupled to a portion of the first conduit, wherein the expandable member is configured to expand against an air passage to inhibit fluid flow past the expandable member;
a second conduit comprising a first end that extends past the expandable member, wherein a portion of the second conduit is positioned external to a lumen of the first conduit and wherein the second conduit is configured to provide for removal of at least one fluid distal to expandable member; and
a third conduit comprising a first end that terminates above the expandable member, wherein a portion of the third conduit is positioned external to a lumen of the first conduit.

2. The airway assembly of claim 1, wherein a portion of the first conduit is partially flexible.

3. The airway assembly of claim 1, wherein reinforcement of the first conduit comprises one or more rings positioned in a body of the first conduit.

4. The airway assembly of claim 1, wherein the first conduit diameter is greater than about 3 mm and less than about 22 mm.

5. The airway assembly of claim 1, wherein the expandable member comprises an inflatable balloon and an inflation conduit.

6. The airway assembly of claim 1, wherein the inflated expandable member blocks the air passage.

7. The airway assembly of claim 1, wherein the second conduit diameter is greater than about 0.1 mm and less than about 3.0 mm.

8. The airway assembly of claim 1, wherein the first end of the second conduit terminates at a distance greater than about 0.01 cm and less than about 0.5 cm from a first end of the first conduit.

9. The airway assembly of claim 1, wherein the second conduit is coupled to an outer surface of the first conduit.

10. The airway assembly of claim 1, further comprising a ventilator configured to couple to the third conduit.

11. The airway assembly of claim 1, further comprising a capnography monitor for CO₂ analysis configured to couple to the third conduit.

12. The airway assembly of claim 1, wherein the third conduit diameter is greater than about 0.1 mm and less than about 3.0 mm.

13. The airway assembly of claim 1, wherein the third conduit is coupled to the outer surface of the first conduit.

14. The airway assembly of claim 1, further comprising a biopsy needle insertable in the lumen of the first conduit.

15. The airway assembly of claim 1, further comprising an endoscope insertable in the lumen of the first conduit.

16. The airway assembly of claim 1, wherein the third conduit is configured to provide ventilation to a patient

17. An airway assembly comprising:
a first conduit comprising reinforcement to inhibit collapse of the first conduit;
an expandable member coupled to a portion of the first conduit, wherein the expandable member is configured to inhibit fluid flow past the expandable member;
a second conduit comprising a first end that extends past the expandable member, wherein the second conduit is coupled to an outer surface of the first conduit; and
a third conduit comprising a first end that terminates before the expandable member, wherein the third conduit is coupled to an outer surface of the first conduit.

18. The airway assembly of claim 17, wherein the first conduit is partially flexible.

19. The airway assembly of claim 17, wherein the reinforcement comprises one or more rings positioned in a body of the first conduit.

20. The airway assembly of claim 17, wherein the expandable member comprises an inflatable balloon.

21. The airway assembly of claim 17, wherein the expandable member comprises an inflation conduit.

22. The airway assembly of claim 17, wherein the first conduit diameter is greater than about 3 mm and less than about 22 mm.

23. The airway assembly of claim 17, wherein the first conduit diameter is greater than about 8.0 mm and less than about 17 mm.

24. The airway assembly of claim 17, wherein the second conduit diameter is greater than about 0.1 mm and less than about 3.0 mm.

25. The airway assembly of claim 17, wherein the second conduit diameter is greater than about than about 0.5 mm and less than about 2.5 mm.

26. The airway assembly of claim 17, wherein the second conduit terminates at a distance greater than about 0.01 cm and less than about 0.5 cm from a distal end of the first conduit.

27. The airway assembly of claim 17, wherein the second conduit is configured to suction bodily fluids and gases distal to the expandable member.

28. The airway assembly of claim 17, wherein the third conduit diameter is greater than about 0.1 mm and less than about 3.0 mm.

29. The airway assembly of claim 17, wherein the third conduit diameter is greater than about 0.5 mm and less than about 2.5 mm.

30. The airway assembly of claim 17, wherein the third conduit is configured to provide a lumen for ventilation.

31. The airway assembly of claim 17, further comprising a ventilator configured to couple to the third conduit.

32. The airway assembly of claim 31, wherein the ventilator comprises a jet ventilator.

33. The airway assembly of claim 17, further comprising a capnography monitor for CO₂ analysis configured to couple to the third conduit.

34. The airway assembly of claim 17, wherein the third conduit is configured to couple to a pressure monitor.

35. The airway assembly of claim 17, further comprising a biopsy needle insertable in the lumen of the first conduit.

36. The airway assembly of claim 17, further comprising an endoscope insertable in the lumen of the first conduit.

37. An airway assembly comprising:
a first conduit comprising reinforcement to inhibit collapse of the first conduit;
an expandable member coupled to a portion of the first conduit, wherein the expandable member is configured to inhibit fluid flow past the expandable member
a second conduit comprising a first end that extends past the expandable member, stops greater than about 0.01 cm and less than about 0.5 mm from the distal end of the first conduit and wherein a portion of the second conduit is coupled to an outer surface of the first conduit; and
a third conduit comprising a first end that terminates proximal the expandable member, wherein a portion of the third conduit is positioned external to a lumen of the first conduit.

38. A method for using an airway assembly comprising:
removing a first conduit, a second conduit, a third conduit and an expandable member from an airway assembly package;
coupling the second conduit, the third conduit, and the expandable member to the first conduit; and
inserting the coupled components in an air passage of a patient.

39. The method of claim 38, wherein an endoscope is inserted in a portion of the first conduit.

40. The method of claim 38, wherein a biopsy needle is inserted in a portion of the first conduit.

41. The method of claim 38, wherein the expandable member comprises an inflation conduit and a balloon cuff configured to block the air passage.

42. A method of using an airway assembly comprising:
inserting a portion of the airway assembly in an air passage;
inflating a portion of the airway assembly against the air passage;
delivering a medication to the air passage through a portion of the airway assembly; and
inserting an instrument in a portion of the airway assembly.
